(19) United States
(12) Patent Application Publication
(10) Pub. No.: US 2022/0280183 A1
(43) Pub. Date: Sep. 8, 2022

(54) ATHERECTOMY DEVICES INCLUDING AXIALLY OSCILLATING CUTTING ELEMENTS

(71) Applicants: PHILIPS IMAGE GUIDED THERAPY CORPORATION, SAN DIEGO, CA (US); KONINKLIJKE PHILIPS N.V., Eindhoven (NL)

(72) Inventors: Douglas ROWE, SAN JOSE, CA (US); Richard LILLY, SAN DIEGO, CA (US); Dino Hamilton DE CICCO, RAMONA, CA (US)

(21) Appl. No.: 17/753,143

(22) PCT Filed: Aug. 21, 2020
(86) PCT No.: PCT/EP2020/073467

§ 371 Date: Feb. 22, 2022

Related U.S. Application Data
(60) Provisional application No. 62/890,114, filed on Aug. 22, 2019.

(51) Int. Cl. A61B 17/3207; A61B 17/32

(52) U.S. Cl. CPC A61B 17/320758; A61B 17/32002; A61B 2017/22038

(57) ABSTRACT

An atherectomy device includes a cutter assembly. The cutter assembly includes a housing, and the housing includes a housing bearing surface. The cutter assembly also includes a cutting element rotatably and translatably carried by the housing. The cutting element includes at least one cutting blade configured to cut occlusive material upon rotation of the cutting element relative to the housing. The cutting element also includes a cutter bearing surface configured to engage the housing bearing surface. Upon the cutting element rotating relative to the housing and the at least one cutting blade cutting occlusive material, the cutting element translates distally relative to the housing until the cutter bearing surface engages the housing bearing surface.

### FIELD OF THE DISCLOSURE

The devices and methods described herein generally relate to treatment of occluded body lumens, such as the removal of occlusive material from a blood vessel or other body parts.

### BACKGROUND

Peripheral and interventional cardiology is a medical specialty that relates to treatment of various forms of cardiovascular disease, including coronary artery disease and peripheral vascular disease. Coronary artery disease and peripheral vascular disease can arise due to the narrowing of the arteries by atherosclerosis (also called arteriosclerosis). Coronary artery disease generally affects arteries of the heart-arteries that carry blood to cardiac muscles and surrounding tissue. Peripheral vascular disease refers to various diseases of the vascular system outside the heart and brain, which carries blood, for example, to the legs.

Atherosclerosis commonly affects the medium and large arteries, and may occur when fat, cholesterol, and other substances build up on the walls of arteries and form fleshy or hard/calcified structures called plaques/lesions. As plaque forms within an arterial wall, the artery may narrow and become less flexible, which may make it more difficult for blood to flow therethrough. In the peripheral arteries, the plaque is typically not localized, but can extend in length along the axis of the artery for as much as 10 mm or more (in some instance up to 400 mm or more).

Pieces of plaque can break off and move through the affected artery to smaller blood vessels, which may in some instances block them and may result in tissue damage or tissue death (embolization). In some cases, the atherosclerotic plaque may be associated with a weakening of the wall of the affected artery, which can lead to an aneurysm. Minimally invasive surgeries may be performed to remove plaque from arteries in an effort to alleviate or help prevent the complications of atherosclerosis.

A number of interventional surgical methodologies may be used to treat atherosclerosis. In balloon angioplasty, for example, a physician may advance a collapsed, intravascular balloon catheter into a narrowed artery, and may inflate the balloon to macerate and/or displace plaque against the vessel wall. A successful angioplasty may help reopen the artery and allow for improved blood flow. Often, balloon angioplasty is performed in conjunction with the placement of a stent or scaffold structure within the artery to help minimize re-narrowing of the artery. Balloon angioplasty, however, can stretch the artery and induce scar tissue formation, while the placement of a stent can cut arterial tissue and also induce scar tissue formation. Scar tissue formation may lead to restenosis of the artery. In some instances, balloon angioplasty can also rip the vessel wall.

Atherectomy is another treatment methodology for atherosclerosis, and involves the use of an intravascular device to mechanically remove (that is, debulk) plaque from the wall of the artery. Atherectomy devices may allow for the removal of plaque from the wall of an artery, reducing the risk of stretching, cutter, or dissecting the arterial wall and causing tissue damage that leads to restenosis. In some instances, atherectomy may be used to treat restenosis by removing scar tissue.

Unfortunately, some atherectomy devices suffer from structural and performance limitations. For example, the cutting elements or assemblies of some atherectomy devices cannot adequately treat total occlusions. Accordingly, it is desirable to provide improved atherectomy devices and methods.

### SUMMARY

The present disclosure presents an atherectomy device. The atherectomy device includes a handle configured to be manipulated by a user. A catheter is coupled to the handle. The catheter includes an outer sheath and a drive shaft, and the drive shaft is disposed within and rotatable relative to the outer sheath. A cutter assembly includes a housing coupled to and extending distally from the outer sheath, and the housing includes a housing bearing surface. The cutter assembly also includes a cutting element rotatably and translatably carried by the housing. The cutting element is coupled to and extends distally from the drive shaft. The cutting element includes at least one cutting blade configured to cut occlusive material upon rotation of the cutting element relative to the housing. The cutting element also includes a cutter bearing surface configured to engage the housing bearing surface. Upon the cutting element rotating relative to the housing and the at least one cutting blade cutting occlusive material, the cutting element translates distally relative to the housing until the cutter bearing surface engages the housing bearing surface.

The atherectomy device according to the previous paragraph, wherein the drive shaft axially elongates as the cutting element translates distally relative to the housing, and whereupon the cutter bearing surface engages the housing bearing surface, the drive shaft axially shortens and translates the cutter element proximally relative to the housing.

The atherectomy device according to any of the previous paragraphs, wherein the cutter assembly further comprises a ferrule coupled to the housing, and whereupon the cutter bearing surface engages the housing bearing surface, the drive shaft axially shortens and translates the cutter element proximally relative to the housing such that the cutter element engages the ferrule.

The atherectomy device according to any of the previous paragraphs, wherein the cutting element is translatable relative to the housing over a distance of 0.010 inches to 0.035 inches.

The atherectomy device according to any of the previous paragraphs, wherein the cutting element is translatable relative to the housing over a distance of 0.015 inches to 0.030 inches.

The atherectomy device according to any of the previous paragraphs, wherein the cutting element is a proximal cutting element, wherein the cutter assembly further comprises a distal cutting element, the distal cutting element being coupled to and rotatable with the proximal cutting element relative to the housing, and the distal cutting element comprises at least one cutting blade.

The atherectomy device according to any of the previous paragraphs, wherein the cutter bearing surface is disposed between a distal end portion and a proximal end portion of the at least one cutting blade.

The present disclosure also presents an atherectomy device. The atherectomy device includes a handle configured to be manipulated by a user. A catheter is coupled to the handle. The catheter includes an outer sheath and a drive shaft, and the drive shaft is disposed within and rotatable relative to the outer sheath. A cutter assembly includes a housing coupled to and extending distally from the outer sheath, and the housing includes a cutter translation chamber. The cutter assembly also includes a cutting element that is rotatably and translatably carried by the housing. The cutting element is coupled to and extends distally from the drive shaft. The cutting element includes at least one cutting blade configured to cut occlusive material upon rotation of the cutting element relative to the housing. The cutting element also includes a cutter restraining portion that is rotatably received in the cutter translation chamber and is translatable from a first position to a second position and vice versa within the cutter translation chamber. Upon the cutting element rotating relative to the housing and the at least one cutting blade cutting occlusive material, the cutter restraining portion rotates and translates from the first position to the second position within the cutter translation chamber, and the cutting element translates distally relative to the housing. Upon the cutter restraining portion reaching the second position, the cutter restraining portion translates from the second position to the first position, and the cutting element translates proximally relative to the housing.

The atherectomy device according to the previous paragraph, wherein the drive shaft axially elongates as the cutting element translates distally relative to the housing, and whereupon the cutter restraining portion reaches the second position, the drive shaft axially shortens and translates the cutter element proximally relative to the housing.

The atherectomy device according to any of the previous paragraphs, wherein the cutting element translates relative to the housing over a distance of 0.010 inches to 0.035 inches as the cutter restraining portion translates from the first position to the second position.

The atherectomy device according to any of the previous paragraphs, wherein the cutting element translates relative to the housing over a distance of 0.015 inches to 0.030 inches as the cutter restraining portion translates from the first position to the second position.

The atherectomy device according to any of the previous paragraphs, wherein the cutting element is a proximal cutting element, wherein the cutter assembly further comprises a distal cutting element, the distal cutting element being coupled to and rotatable with the proximal cutting element relative to the housing, and the distal cutting element comprises at least one cutting blade.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together. When each one of A, B, and C in the above expressions refers to an element, such as X, Y, and Z, or class of elements, such as X₁-Xₙ, Y₁-Yₘ, and Z₁-Zₒ, the phrase is intended to refer to a single element selected from X, Y, and Z, a combination of elements selected from the same class (for example, X₁ and X₂) as well as a combination of elements selected from two or more classes (for example, Y₁ and Zₒ).

The term “a” or “an” entity refers to one or more of that entity. As such, the terms “a” (or “an”), “one or more” and “at least one” may be used interchangeably herein. It is also to be noted that the terms “comprising”, “including”, and “having” may be used interchangeably.

The term “means” as used herein shall be given its broadest possible interpretation in accordance with 35 U.S.C. Section 112(f). Accordingly, a claim incorporating the term “means” shall cover all structures, materials, or acts set forth herein, and all of the equivalents thereof. Further, the structures, materials or acts and the equivalents thereof shall include all those described in the summary, brief description of the drawings, detailed description, abstract, and claims themselves.

It should be understood that every maximum numerical limitation given throughout this disclosure is deemed to include each and every lower numerical limitation as an alternative, as if such lower numerical limitations were expressly written herein. Every minimum numerical limitation given throughout this disclosure is deemed to include each and every higher numerical limitation as an alternative, as if such higher numerical limitations were expressly written herein. Every numerical range given throughout this disclosure is deemed to include each and every narrower numerical range that falls within such broader numerical range, as if such narrower numerical ranges were all expressly written herein.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

### BRIEF DESCRIPTION OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure may be made and used and are not to be construed as limiting the disclosure to only the illustrated and described examples. Further features and advantages will become apparent from the following, more detailed, description of the various aspects, embodiments, and configurations of the disclosure, as illustrated by the drawings referenced below.

FIG. 1 is a side view of an atherectomy system according to an embodiment of the present disclosure.

FIG. 2A is a detail side view of a distal portion of the atherectomy system of FIG. 1.

FIG. 2B is a detail perspective view of the distal portion of the atherectomy system of FIG. 1.

FIG. 2C is a detail transverse sectional view of the distal portion of the atherectomy system of FIG. 2A.

FIG. 3A is a detail side view of the distal portion of the atherectomy system of FIG. 2A with a housing shown in hidden lines and cutting elements shown in a first position relative to the housing.

FIG. 3B is a detail side view of the distal portion of the atherectomy system of FIG. 2A with the housing shown in hidden lines and the cutting elements shown in a second position relative to the housing.

FIG. 4A is a perspective view of a distal cutting element of the atherectomy system of FIG. 1.

FIG. 4B is a side view of the distal cutting element of FIG. 4A.

FIG. 4C is a cross sectional view of the distal cutting element along line 4C-4C of FIG. 4B.

FIG. 5A is a perspective view of a proximal cutting element of the atherectomy system of FIG. 1.

FIG. 5B is a front view of the proximal cutting element of FIG. 5A.

It should be understood that the drawings are not necessarily to scale. In certain instances, details that are not necessary for an understanding of the disclosure or that render other details difficult to perceive may have been omitted. It should be understood, of course, that the disclosure is not necessarily limited to the particular embodiments illustrated herein.

### DETAILED DESCRIPTION

Before any embodiments of the disclosure are explained in detail, it is to be understood that the disclosure is not limited in its application to the details of construction and the arrangement of components set forth in the following description or illustrated in the following drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

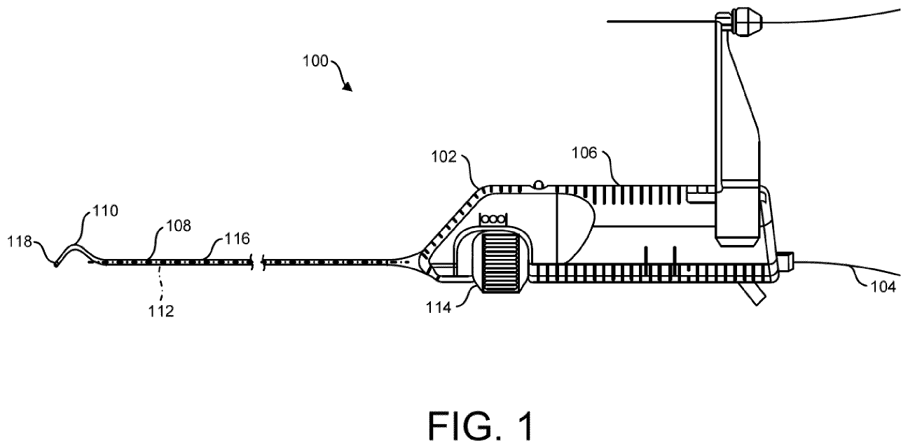

The present disclosure relates generally to devices, systems, and methods for mechanical atherectomy. Referring to FIG. 1, there is shown an exemplary embodiment of the atherectomy systems described herein. The atherectomy system 100 includes an intravascular atherectomy device 102 and a guide wire 104 over which the atherectomy device 102 may be deployed. In some embodiments, the guide wire 104 is silicon-coated or non-coated (bare), or otherwise free of a PTFE coating. Atherectomy systems according to some embodiments of the present disclosure comprise a guide wire 104 that includes a PTFE coating, or atherectomy systems according to some embodiments of the present disclosure lack a guide wire 104.

With continued reference to FIG. 1, the atherectomy device 102 generally includes a handle 106 and a catheter 108. The handle 106 is configured to be grasped and manipulated by a user (for example, a medical professional) during an atherectomy procedure. The catheter 108 is coupled to and extends distally relative to the handle 106. The catheter 108 is configured to be positioned in the vasculature of a subject (for example, a patient) during an atherectomy procedure to facilitate removal of occlusive material (for example, plaque) therefrom. In some embodiments and as illustrated, a distal portion 110 of the catheter 108 has a curved shape or configuration. In some embodiments, the distal portion 110 of the catheter 108 normally has a curved configuration (“normally” being understood as the catheter 108 not being subjected to any external contact forces due to, for example, contact with blood vessel walls) and may be deflected to other configurations. In other embodiments, the distal portion 110 of the catheter 108 normally has a straight shape or configuration and may be deflected to other configurations. In some embodiments, the catheter 108 is selectively rotatable about a catheter rotational axis 112 relative to the handle 106 to facilitate appropriately positioning and or “sweeping” the distal portion 110 of the catheter 108 during an atherectomy procedure. In some embodiments and as illustrated, the handle 106 carries a rotatable knob or dial 114 for selectively rotating the catheter 108 relative to the handle 106. The catheter 108 includes an outer sheath 116, and the outer sheath 116 couples to a cutter assembly 118 that extends distally therefrom. The cutter assembly 118 is described in further detail below.

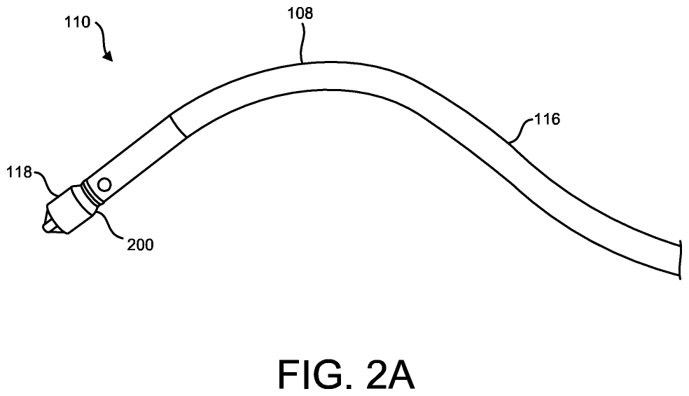

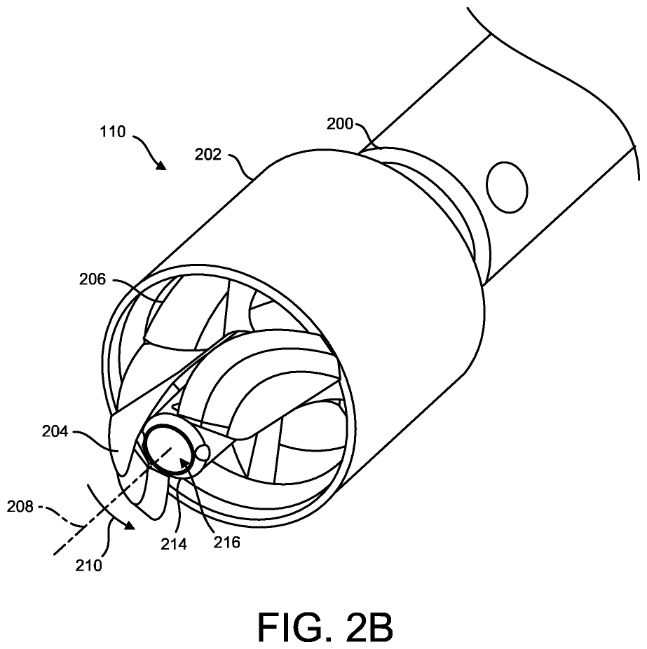

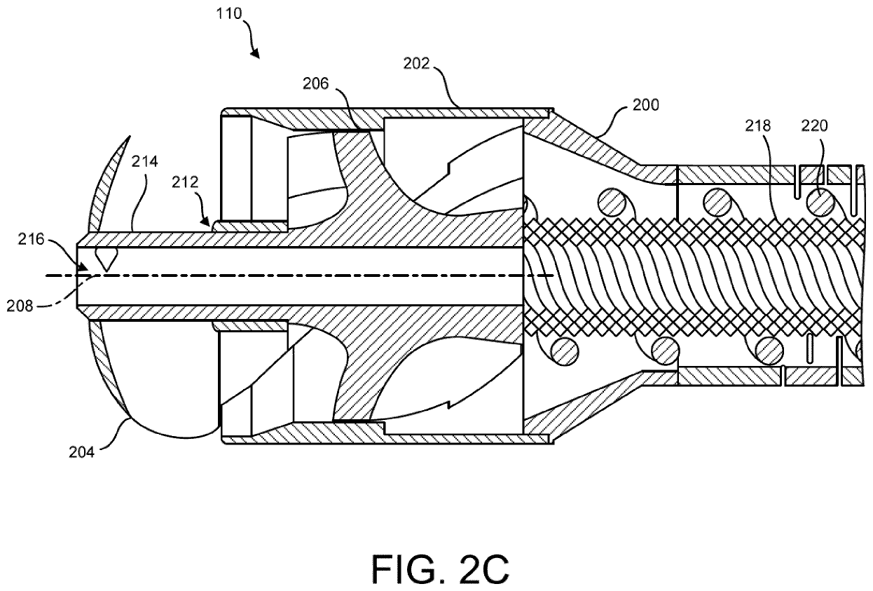

FIGS. 2A-2C illustrate the distal portion 110 of the catheter 108, including, among other components, the outer sheath 116 and the cutter assembly 118. The cutter assembly 118 includes a ferrule 200 that couples to the outer sheath 116 and extends distally therefrom. The cutter assembly 118 further includes a housing 202 that couples to the ferrule 200 and extends distally therefrom. The housing 202 rotatably carries cutting elements. Referring specifically to FIGS. 2B-2C, the housing 202 rotatably carries a first, or distal, cutting element 204 and a second, or proximal, cutting element 206. Rotation of the first cutting element 204 and the second cutting element 206 about a rotation axis 208 in a rotational direction 210 relative to the housing 202 causes the cutting elements 204, 206 to cut occlusive material and convey the occlusive material into the housing 202 (a process also referred to as “debulking”).

Still referring to FIGS. 2B-2C, the first cutting element 204 generally extends distally from the second cutting element 206 and the housing 202. The first cutting element 204 includes a central opening 212 (see FIG. 2C) for coupling to the second cutting element 206. The second cutting element 206 is generally disposed within the housing 202 and, in some embodiments and as illustrated, may be completely disposed within the housing 202. The second cutting element 206 is also generally disposed proximally from the first cutting element 204, although the second cutting element 206 includes a shaft or stem 214 that is received in the central opening 212. The stem 214 may couple to the first cutting element 204 in various manners. For example, the stem 214 may couple to the first cutting element 204 via welding. In some embodiments and as illustrated, the stem 214 extends distally relative to the first cutting element 204. The stem 214 includes an inner lumen 216 for receiving a guide wire (shown elsewhere).

Referring specifically to FIG. 2C, the atherectomy device 102 further includes a rotatable drive shaft 218 that couples the first cutting element 204 and the second cutting element 206 to a prime mover (for example, a motor carried by the handle 106 —not shown). That is, the prime mover rotates the drive shaft 218, which in turn rotates the first cutting element 204 and the second cutting element 206 to facilitate cutting occlusive material and conveying the occlusive material into the housing 202. In some embodiments, the cutter assembly 118 captures the cut occlusive material from the blood without the use of vacuum aspiration. In other embodiments, vacuum aspiration may assist capture of the cut occlusive material.

With continued reference to FIG. 2C, in some embodiments, the atherectomy device 102 also includes an internal conveyor 220 that is coupled to and rotates with the drive shaft 218. As occlusive material is conveyed into the cutter housing 202 by the first cutting element 204 and the second cutting element 206, the conveyor 220 displaces the cut occlusive material proximally through the catheter 108 for discharge outside the subject's body. In some embodiments, this conveyance may occur without the use of vacuum aspiration assistance. In other embodiments, vacuum aspiration may assist conveyance of the cut occlusive material.

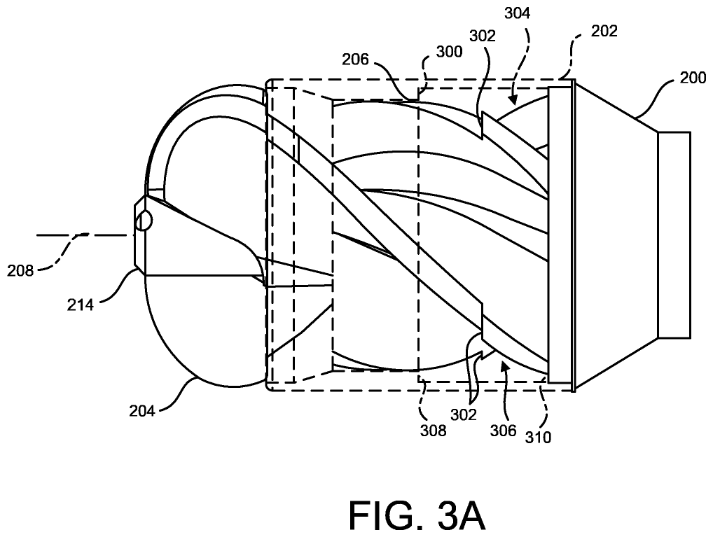

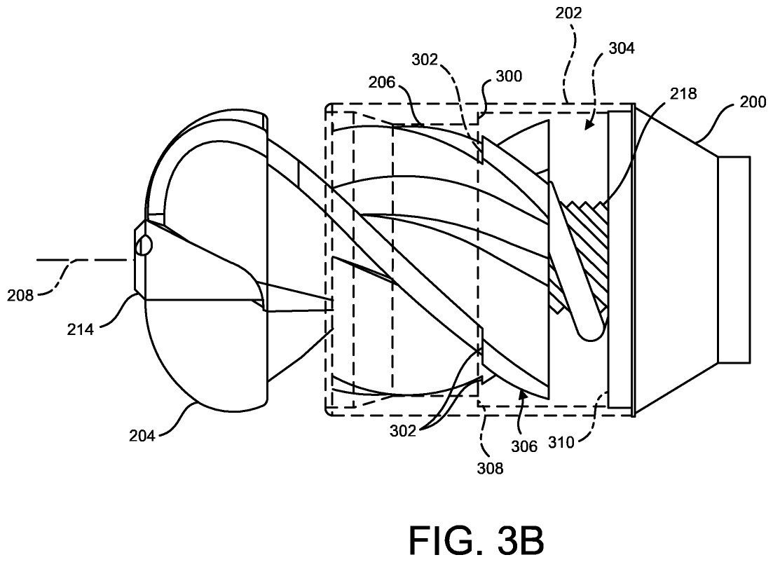

The cutter assembly 118 includes features that permit the cutting elements 204, 206 to repeatedly translate distally and proximally, or translatably oscillate, relative to the housing 202 during an atherectomy procedure. In some situations, such motion causes the cutting elements 204, 206 to make relatively small interrupted cuts in occlusive material. This facilitates efficient cutting and conveying relatively small amounts of occlusive material, which in turn reduces the likelihood of clogging and potentially provides more effective cutting of relatively hard occlusive materials, such as calcium. More specifically and referring to FIGS. 3A and 3B, the housing 202 includes an internal housing bearing surface 300, and the second cutting element 206 includes one or more cutter bearing surfaces 302 (illustratively, four cutter bearing surfaces 302, three of which are visible in FIGS. 3A and 3B). When the cutter assembly 118 is advanced relative to occlusive material and the cutting elements 204, 206 rotate relative to the housing 202 and cut occlusive material, the cutting elements 204, 206 convey the occlusive material proximally as described above. This action urges the cutting elements 204, 206 to move distally. That is, this action causes the cutting elements 204, 206 to translate from the position shown in FIG. 3A to the position shown in FIG. 3B, and the drive shaft 218 axially elongates or stretches. When the cutting elements 204, 206 reach the position shown in FIG. 3B, the cutter bearing surfaces 302 engage the housing bearing surface 300, which inhibits the cutting elements 204, 206 from further translating distally relative to the housing 202. As a result, and provided that the cutter assembly 118 is being advanced at a sufficiently low rate (for example, 1 mm/sec or less), the cutting elements 204, 206 are inhibited from cutting and proximally conveying further occlusive material. Accordingly, the cutting elements 204, 206 are not urged distally by occlusive material, the drive shaft 218 axially shortens or relaxes, and the cutting elements 204, 206 translate from the position shown in FIG. 3B to the position shown in FIG. 3A. When the cutting elements 204, 206 reach the position shown in FIG. 3A, the second cutting element 206 engages the ferrule 200. The process then repeats until termination of advancement of the cutter assembly 118 or rotation of the cutting elements 204, 206.

The cutting elements 204, 206 may translate various distances relative to the housing 202 during the above process. In some embodiments, the cutting elements 204, 206 translate a distance of 0.010 inches to 0.035 inches relative to the housing 202 during the above process. In some embodiments, the cutting elements 204, 206 translate a distance of 0.015 inches to 0.030 inches relative to the housing 202 during the above process.

The features of the cutter assembly 118 that permit the cutting elements 204, 206 to repeatedly translatably oscillate relative to the housing 202 may be described in other manners. For example, the housing 202 may be described as including an internal cutter translation chamber 304, and the second cutting element 206 may be described as including a cutter restraining portion 306 that is rotatably received in the cutter translation chamber 304.

The cutter restraining portion 306 is translatable from a first position (more specifically, as shown in FIG. 3A) to a second position (more specifically, as shown in FIG. 3B) and vice versa with the cutter translation chamber 304. When the cutter assembly 118 is advanced relative to occlusive material and the cutting elements 204, 206 rotate relative to the housing 202 and cut occlusive material, the cutting elements 204, 206 convey the occlusive material proximally as described above. This action urges the cutting elements 204, 206 to move from the first position toward the second position, and the drive shaft 218 axially elongates or stretches. When the cutting elements 204, 206 reach the second position, the cutter restraining portion 306 reaches a distal end portion 308 of the cutter translation chamber 304, which inhibits the cutting elements 204, 206 from further translating distally relative to the housing 202. As a result, and provided that the cutter assembly 118 is being advanced at a sufficiently low rate (for example, 1 mm/sec or less), the cutting elements 204, 206 are inhibited from cutting and proximally conveying further occlusive material. Accordingly, the cutting elements 204, 206 are not urged distally by occlusive material, the drive shaft 218 axially shortens or relaxes, and the cutting elements 204, 206 translate from the second position to the first position. When the cutting elements 204, 206 reach the first position, the second cutting element 206 reaches a proximal end portion 310 of the cutter translation chamber 304. The process then repeats until termination of advancement of the cutter assembly 118 or rotation of the cutting elements 204, 206.

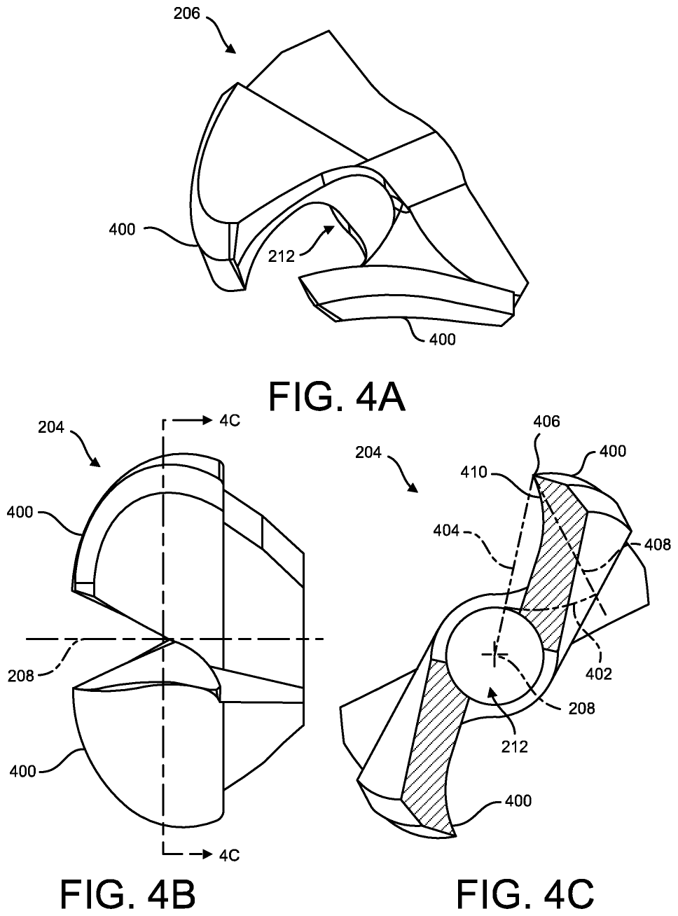

Referring now to FIGS. 4A-4C, the first cutting element 204 includes one or more first, or distal, cutting flutes or blades that extend distally relative to the housing 202. In some embodiments and as illustrated, the first cutting element 204 includes two distal cutting blades 400. In some embodiments, the first cutting element 204 includes a different number of cutting blades, such as three, four, five, six, seven, eight, nine, ten, or more cutting blades. In some embodiments and as illustrated, one or more of the distal cutting blades 400 extend helically relative to the rotation axis 208 of the first cutting element 204 and the second cutting element 206.

Referring specifically to FIG. 4C, in some embodiments the distal cutting blades 400 have a positive rake angle 402. That is, the distal cutting blades 400 have a rake angle 402 that is measured between an imaginary radius 404 extending from the rotation axis 208 of the first cutting element 204 to a most radially distant edge 406 of the cutting blade 400 and a tangent 408 from an inner face 410 of the cutting blade 400 at the most radially distant edge 406. The rake angle 402 is in the same direction as the rotational direction 210 of the first cutting element 204 and the second cutting element 206 about the rotation axis 208. In some embodiments, the rake angle 402 is in a range of 40 degrees to 80 degrees. In some embodiments, the rake angle 402 is in a range of 45 degrees to 75 degrees. In some embodiments, the rake angle 402 is in a range of 40 degrees to 70 degrees. In some embodiments, the rake angle 402 is in a range of 45 degrees to 65 degrees. In some embodiments, the rake angle 402 is in a range of 50 degrees to 60 degrees. In some embodiments, the rake angle 402 is substantially 55 degrees (that is, 55 degrees±2.5 degrees).

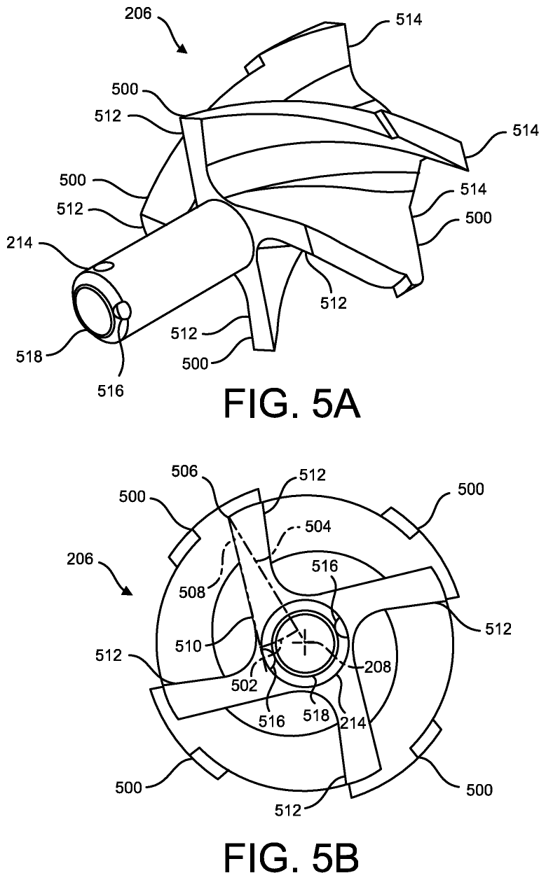

Referring now to FIGS. 5A-5B, the second cutting element 206 includes one or more second, or proximal, cutting flutes or blades 500. In some embodiments, the second cutting element 206 has two times the number of blades 500 as the first cutting element 204. In some embodiments and as illustrated, the second cutting element 206 includes four cutting blades 500. In some embodiments and as illustrated, the proximal cutting blades 500 extend helically relative to the rotation axis 208 of the first cutting element 204 and the second cutting element 206. In some embodiments and referring specifically to FIG. 5B, the proximal cutting blades 500 have a negative rake angle 502. Stated another way, the proximal cutting blades 500 have a rake angle 502 that is measured between an imaginary radius 504 extending from the rotation axis 208 of the second cutting element to a most radially distant edge 506 of the cutting blade 500 to a tangent 508 from an inner face 510 of the cutting blade 500 at the most radially distant edge 506. The rake angle 502 is in the opposite direction as the rotational direction 210 of the first cutting element 204 and the second cutting element 206 about the rotation axis 208. Stated yet another way, the inner face 510 the proximal cutting blades 500 may slant outward or forward of the cutting edge. In some embodiments, the rake angle 502 is in a range of 5 degrees to 45 degrees (also referred to as −5 degrees to −45 degrees). In some embodiments, the rake angle 502 is in a range of 6 degrees to 35 degrees (also referred to as -−6degrees to −35 degrees). In some embodiments, the rake angle 502 is in a range of 8 degrees to 24 degrees (also referred to as −8 degrees to −24 degrees). In some embodiments, the rake angle 502 is substantially 16 degrees (that is, 16 degrees±2.5 degrees; also referred to as substantially −16 degrees (that is, −16 degrees±2.5 degrees)).

In some embodiments, the positive rake angle 402 of the first cutting element 204 and the negative rake angle 502 of the second cutting element 206 facilitate improved cutting efficiency and/or inhibit clogging of occlusive material in the housing 202. More specifically, in some embodiments the positive rake angle 402 of the first cutting element 204 facilitates cutting and conveying occlusive material toward the second cutting element 206 and the negative rake angle 502 of the second cutting element 206 facilitates displacing occlusive materially radially outwardly toward the housing 202 and proximally, thereby inhibiting clogging of occlusive material in the housing 202.

In some embodiments and as illustrated, the cutter bearing surfaces 302 are formed on each of the proximal cutting blades 500. In some embodiments and as illustrated, the cutter bearing surfaces 302 may be disposed between distal end portions 512 and proximal end portions 514 of the proximal cutting blades 500. In such embodiments, the cutter bearing surfaces 302 may be disposed at any of various locations between distal end portions 512 and proximal end portions 514 of the proximal cutting blades 500. In some embodiments, the cutter bearing surfaces 302 are disposed at other locations, such as the distal end portions 512 of the proximal cutting blades 500.

In some embodiments and as illustrated, the cutting stem 214 also includes one or more cutting features 516 that facilitate fragmenting occlusive material into small particles to be captured and removed by the atherectomy system 100. In some embodiments and as illustrated, the cutting stem 214 includes two cutting features 516. In other embodiments, the cutting stem 214 includes a different number of cutting features 516 (for example, one, three, four, five, six, seven, eight, nine, ten, or more cutting features 516). In some embodiments, the cutting features 516 are negative features (for example, depressions formed on the surface of the cutting stem 214, as illustrated, or channels formed on the surface of the cutting stem 214). In some embodiments, the cutting features 516 are positive features (for example, ridges or protrusions extending from the surface of the cutting stem 214). In some embodiments, the cutting features 516 extend substantially proximally from a leading end 518 of the cutting stem 214. In some embodiments, the cutting features 516 are disposed apart from the leading end 518 and/or do not extend proximally along the stem 214.

The foregoing discussion has been presented for purposes of illustration and description. The foregoing is not intended to limit the disclosure to the form or forms disclosed herein. In the foregoing Summary for example, various features of the disclosure are grouped together in one or more aspects, embodiments, and/or configurations for the purpose of streamlining the disclosure. The features of the aspects, embodiments, and/or configurations of the disclosure may be combined in alternate aspects, embodiments, and/or configurations other than those discussed above. This method of disclosure is not to be interpreted as reflecting an intention that the claims require more features than are expressly recited in each claim. Rather, as the following claims reflect, inventive aspects lie in less than all features of a single foregoing disclosed aspect, embodiment, and/or configuration. Thus, the following claims are hereby incorporated into this Detailed Description, with each claim standing on its own as a separate preferred embodiment of the disclosure.

Moreover, though the description has included description of one or more aspects, embodiments, and/or configurations and certain variations and modifications, other variations, combinations, and modifications are within the scope of the disclosure, for example, as may be within the skill and knowledge of those in the art, after understanding the present disclosure. It is intended to obtain rights which include alternative aspects, embodiments, and/or configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. An atherectomy device, comprising:
a handle configured to be manipulated by a user;
a catheter coupled to the handle, the catheter comprising an outer sheath and a drive shaft, wherein the drive shaft is disposed within and rotatable relative to the outer sheath; and
a cutter assembly comprising:
a housing coupled to and extending distally from the outer sheath, the housing comprising a housing bearing surface;
a cutting element rotatably and translatably carried by the housing, the cutting element being coupled to and extending distally from the drive shaft, the cutting element comprising:
at least one cutting blade configured to cut occlusive material upon rotation of the cutting element relative to the housing; and
a cutter bearing surface configured to engage the housing bearing surface;
whereupon the cutting element rotates relative to the housing and the at least one cutting blade cuts occlusive material, the cutting element translates distally relative to the housing until the cutter bearing surface engages the housing bearing surface.

2. The atherectomy device of claim 1, wherein the drive shaft axially elongates as the cutting element translates distally relative to the housing, and whereupon the cutter bearing surface engages the housing bearing surface, the drive shaft axially shortens and translates the cutter element proximally relative to the housing.

3. The atherectomy device of claim 1, wherein the cutter assembly further comprises a ferrule coupled to the housing, and whereupon the cutter bearing surface engages the housing bearing surface, the drive shaft axially shortens and translates the cutter element proximally relative to the housing such that the cutter element engages the ferrule.

4. The atherectomy device of claim 1, wherein the cutting element is translatable relative to the housing over a distance of 0.010 inches to 0.035 inches.

5. The atherectomy device of claim 1, wherein the cutting element is translatable relative to the housing over a distance of 0.015 inches to 0.030 inches.

6. The atherectomy device of claim 1, wherein the cutting element is a proximal cutting element, wherein the cutter assembly further comprises a distal cutting element, the distal cutting element being coupled to and rotatable with the proximal cutting element relative to the housing, and the distal cutting element comprises at least one cutting blade.

7. The atherectomy device of claim 1, wherein the cutter bearing surface is disposed between a distal end portion and a proximal end portion of the at least one cutting blade.

8. An atherectomy device, comprising:
a handle configured to be manipulated by a user;
a catheter coupled to the handle, the catheter comprising an outer sheath and a drive shaft, wherein the drive shaft is disposed within and rotatable relative to the outer sheath; and
a cutter assembly comprising:
a housing coupled to and extending distally from the outer sheath, the housing comprising a cutter translation chamber;
a cutting element rotatably and translatably carried by the housing, the cutting element being coupled to and extending distally from the drive shaft, the cutting element comprising:
at least one cutting blade configured to cut occlusive material upon rotation of the cutting element relative to the housing; and
a cutter restraining portion rotatably received in the cutter translation chamber and being translatable from a first position to a second position and vice versa within the cutter translation chamber;
whereupon the cutting element rotates relative to the housing and the at least one cutting blade cuts occlusive material, the cutter restraining portion rotates and translates from the first position to the second position within the cutter translation chamber, and the cutting element translates distally relative to the housing, and whereupon the cutter restraining portion reaches the second position, the cutter restraining portion translates from the second position to the first position, and the cutting element translates proximally relative to the housing.

9. The atherectomy device of claim 8, wherein the drive shaft axially elongates as the cutting element translates distally relative to the housing, and whereupon the cutter restraining portion reaches the second position, the drive shaft axially shortens and translates the cutter element proximally relative to the housing.

10. The atherectomy device of claim 8, wherein the cutting element translates relative to the housing over a distance of 0.010 inches to 0.035 inches as the cutter restraining portion translates from the first position to the second position.

11. The atherectomy device of claim 8, wherein the cutting element translates relative to the housing over a distance of 0.015 inches to 0.030 inches as the cutter restraining portion translates from the first position to the second position.

12. The atherectomy device of claim 8, wherein the cutting element is a proximal cutting element, wherein the cutter assembly further comprises a distal cutting element, the distal cutting element being coupled to and rotatable with the proximal cutting element relative to the housing, and the distal cutting element comprises at least one cutting blade.
